(19) United States
(12) Patent Application Publication
(10) Pub. No.: US 2025/0170414 A1
(43) Pub. Date: May 29, 2025

(54) METHOD OF INTERVERTEBRAL DISC RECOVERY IN INTERVERTEBRAL HERNIA

(71) Applicants: SINDBAD GROUP LIMITED, Hong Kong (CN); IMBAMED SIA, RÌga (LV)

(72) Inventors: Ivan Andreevich PAKHOMOV, Vzevolozhskij rajon (RU); Andrej Georgievich PAKHOMOV, Vzevolozhskij rajon (RU); Ivan Surenovich SARDARYAN, Saint-Petersburg (RU)

(21) Appl. No.: 18/724,881

(22) PCT Filed: Dec. 13, 2022
(86) PCT No.: PCT/IB2022/062159

§ 371 Date: Jun. 27, 2024

(30) Foreign Application Priority Data
Dec. 27, 2021 (EA) 202193286

(51) Int. Cl. A61N 5/06; A61N 5/067

(52) U.S. Cl. CPC A61N 5/0601; A61N 5/067; A61N 2005/0602; A61N 2005/0612; A61N 2005/0663

(57) ABSTRACT

The disclosure provides a method of treating intervertebral disc hernia, the method including applying low intensity laser radiation to the soft tissues of the patient, surrounding the hernia. The disclosure also provides a radiating system for implementing the method and use of the radiating system treating intervertebral disc hernia.

### FIELD

The disclosure generally relates to medicine, namely to laser therapy, traumatology and neurosurgery. In particular, the disclosure relates to a method of treatment of intervertebral disc hernia using laser radiation.

### BACKGROUND

Injuries to the human spine and subsequent pain are one of the most prevalent debilitating conditions affecting the human population. For many of those affected, no position can ease the pain or discomfort associated with spinal injuries or deformities. Such spine related pain can lead to decreased productivity due to loss of work hours, addiction to pain-killing drugs, emotional distress, and prolonged hospital stays. One common cause for many instances of chronic pain is the bulging, or herniation of the intervertebral disc.

The intervertebral disc is made of two parts, a tough collagen outer layer, known as the annulus fibrosus (hereinafter also referred to as “annulus”), and a soft central core known as the nucleus pulposus. The annulus is composed of numerous concentric rings or layers of fibrocartilaginous tissue. The nucleus is a gelatinous material, which forms the center of the disc. The discs tend to vary in size and shape with their position in the spine. The nucleus is composed of a loose, nonoriented, collagen fibril framework supporting a network of cells resembling fibrocytes and chondrocytes. This entire structure is embedded in a gelatinous matrix of various glucosaminoglycans, water, and salts. This material is usually under considerable pressure and is restrained by the annulus.

A tear or weakening in the layers of the annulus fibrosus portion of the disc can allow the soft center portion of the disc (the nucleus) to leak out of the annulus, alternatively, the weakened annulus may simply bulge. A ruptured disc may allow the leaking nucleus pulposus material to press up against a spinal nerve root or spinal cord, causing pain, numbness, tingling and/or weakness in a person's extremities. Herniated discs may occur at any level of the spine, but are more common in the lumbar area, followed in frequency of occurrence by the thoracic region and cervical region. Weakening or tearing of the annulus fibrosus may also result in bulging of the annulus fibrosus due to pressure of the nucleus pulposus against the annulus. The bulging tissue may also impinge upon the nerve root or spinal column, causing pain.

In addition, hernia may often occur as a result of natural ageing of the vertebra. As people age, discs start to become dry and weak, discs shrink and the distance between the discs decrease. Morphological changes lead to the dehydration of nucleus pulposus, disruption, rupture and tearing of the collagen structure of annulus fibrosus, and leakage of nucleus pulposus. This process is called disc degeneration. The reasons for degeneration include age, disease and trauma, as a result of which intervertebral disc loses its elasticity, functionality and causes pain pressuring the nerves.

Laser vaporization is a common approach in treating intervertebral disc hernia and osteochondrosis. Laser vaporization of an intervertebral disc hernia is a minimally invasive method of removing a hernia formation by applying current to it or a laser beam, which leads to heating and gradual evaporation of soft and cartilage tissues and to a decrease in pathological formation. In particular, UA 92681 discloses a method of surgical treatment of protrusions and non-sequestration of hernias of the intervertebral disc, which includes a puncture laser vaporization of the intervertebral disc. RU 2212916 discloses an analogue of laser vaporization. In particular, the document describes a method of treatment of osteochondrosis of the spine, comprising access to the nucleus pulposus of the intervertebral disc and the formation of a cavity inside it by evaporation of the core material using a laser.

Although the method of laser vaporization is common, it has a disadvantage in that after some time, the hernia will begin to increase again and repeated surgery will be required to remove it. This method has also other shortcomings. For example, it cannot be applied to the hernias of size more than 6 mm. Furthermore, laser vaporization method has also some counter indications such as: risk of the spinal cord damage as the hernia puts pressure on the spinal cord, which can be damaged during the procedure; the presence of spondylosis and similar disorders; prolapse of the nucleus pulposus; defeat by the pathological formation of roots of nerve endings; age group—from 50 years and older; the presence of degenerative stenosis; disc ossification. Laser vaporization provides only short-term relief from pain. Relief occurs immediately, but after a while the pain returns again. This is due to the fact that the vaporization of tissues leads to eliminating the signs of hernia only, and does not affect the causes of its development. Furthermore, vaporization leads to the gradual destruction of the cartilage structures of the intervertebral disc. After heating the disc, it loses most of the fluid, which provokes development of degenerative processes. A disc that was subjected to vaporization will no longer be able to return to its previous support function. Therefore, the patient will again feel pain caused by a hernia.

In addition, a recovery after laser vaporization is necessary. It is strictly forbidden for the patient to perform the following actions: to go in for sports, including swimming; to ride a bike; to sit for a long time; to be in a pose that implies a load on the back for a long time (work in the garden) up to 2 weeks after the laser vaporization. In order to prevent development of any complications, it is recommended to take non-steroidal anti-inflammatory drugs within 2 weeks after the operation using the laser vaporization; and the patient should wear a tight corset for a month.

Laser radiation is also often used in the treatment of degenerative disc disease. In particular, the laser radiation may be applied externally; to the epidural cavity; or to the disc and the nucleus pulposus. Specifically, RU 2277393 discloses application of laser to the epidural cavity, i.e. the vertebral canal. WO 2007/038004 describes a method of treating degenerative disc disease by laser radiation of the annulus fibrosis and nucleus pulposus. These methods use direct application of the laser radiation to the herniated disc that may be dangerous to patient's health.

Thus, there is still need for improved method of treating intervertebral disc hernias without surgery and direct impact on the disc, a method that is easy to implement and that provides faster recovery of the patient after the treatment and that does not cause a relapse.

### SUMMARY

In one aspect, the present disclosure provides a method of treating a hernia of an intervertebral disc in a patient, the method comprising introducing at least one light guide into the patient's soft tissue, surrounding the hernia, and subjecting the soft tissue to a laser radiation using said light guide, the laser radiation having a wavelength in the range of from 625 to 740 nm and a power in the range of from 1 and 100 mW.

The disclosed method allows treatment of a hernia of the intervertebral disc using low-intensity laser radiation, without directly impacting the hernia, with simultaneous plastic repair (filling) of the hernial canal with local tissues. This method allows treating an intervertebral disc hernia of any region of the vertebral column, any location, any diameter, including the sequester.

In one embodiment the wavelength of the laser radiation is from 630 to 700 nm, preferably from 630 to 680 nm, more preferably from 630 to 650 nm.

In another embodiment the power of the laser radiation is from 1 to 50 mW, preferably from 3 to 20 mW, more preferably from 6 to 15 mW.

The increased power of the laser radiation provides faster treatment of a hernia of the intervertebral disc, but at the same time increases the risk of negative impact (for example, burn).

The intensity within the indicated ranges provide a balance between the benefits and potential risks.

In yet another embodiment, the time of subjecting the soft tissue to the laser radiation is from 1 to 500 min, preferably from 30 to 100 min, more preferably from 60 to 100 min.

In yet another embodiment, the light guide is introduced into the soft tissue in the plane of the intervertebral disc being treated±0.5 cm, in the anterior direction at a distance from 0.5 to 1.5 cm away from the intervertebral joints.

In yet another embodiment, the light guide is introduced into the soft tissue at a distance from 1.5 to 2.5 cm away from the midsagittal plane passing through the spinous processes.

In yet another embodiment, the soft tissue is the muscle tissue.

In yet another embodiment, the muscle tissue is the tissue of the dorsal paravertebral muscles.

In yet another embodiment, the light guide is introduced into the tissue of the dorsal paravertebral muscles at a distance at least 1.0-1.5 cm away from the border of the dorsal paravertebral muscles with the thoracolumbar fascia.

The introduction of the light guide into the specified location provides further improvement in the effectiveness of the treatment of the hernia using laser radiation.

In yet another embodiment more than one hernias are subjected to laser radiation simultaneously, in particular from 2 to 4 hernias are subjected to laser radiation simultaneously.

The possibility of simultaneous subjecting to laser radiation of several hernias provides a reduction of time necessary for the treatment compared with the sequential subjecting of the hernias.

In yet another embodiment, the at least one light guide is introduced at both sides of the midsagittal plane.

In yet another embodiment, a single hernia is subjected to the laser radiation using more than one light guide, in particular, from 2 to 8 light guides.

In yet another embodiment, the steps of the proposed method are repeated several times, in particular, from 10 to 40 times, 3-7 times a week.

Applying several light guides to a single hernia accelerates the treatment process.

In yet another embodiment, the method further comprises a step of indirect intravascular laser blood irradiation.

The step of indirect intravascular laser blood irradiation provides acceleration of the treatment process.

In a particular embodiment, the step of indirect intravascular laser blood irradiation is carried out by subjecting a carrier solution to a laser radiation and simultaneously introducing the carrier solution being irradiated into the patient's vascular system.

In yet another particular embodiment, the laser radiation at the step of indirect intravascular laser blood irradiation has a wavelength in the range of from 625 to 740 nm and a power in the range of from 1 to 15 mW and is applied during a time period of from 5 to 120 min.

In yet another particular embodiment, the carrier solution comprises NaCl, nicotinate, vitamin C, and water.

In yet another particular embodiment, the step of indirect intravascular laser blood irradiation is repeated several times, in particular, from 2 to 7 times, once in 5-10 days.

In yet another embodiment, the method further comprises a step of direct intravascular laser blood irradiation.

The step of direct intravascular laser blood irradiation provides acceleration of the treatment process.

In a particular embodiment, the step of indirect intravascular laser blood irradiation is carried out by directly subjecting the patient's blood to a laser radiation.

In yet another particular embodiment, the laser radiation at the step of direct intravascular laser blood irradiation has a wavelength in the range of from 625 to 740 nm and a power in the range of from 1 to 15 mW, more preferably in the range of from 1 to 7 mW, even more preferably in the range of from 1.5 to 3 mW, and is applied during a time period of from 5 to 120 min.

In yet another particular embodiment, the step of direct intravascular laser blood irradiation is repeated several times, in particular, from 2 to 7 times, once in 5-10 days.

The proposed method is useful for treating hernia of any size.

In a particular embodiment the hernia has a size in the range of from 1 to 40 mm.

In yet another embodiment, the hernia of intervertebral disc is a sequestered hernia of the intervertebral disc.

In a particular embodiment, the light guide is introduced inside a needle.

In another aspect, the present disclosure provides a radiating system for treating a hernia of an intervertebral disc in a patient, the system comprising:
a radiating source operable to generate a laser radiation having a wavelength in the range of from 625 to 740 nm; wherein
the radiating source is configured to be connected with at least one light guide for delivering the laser radiation into the patient's soft tissue, surrounding the hernia, with a power in the range of from 1 and 100 mW.

In a particular embodiment of the radiating system of this aspect, the power of the laser radiation delivered into the patient's soft tissue is from 1 to 50 mW, preferably from 3 to 20 mW, more preferably from 6 to 15 mW.

In another aspect, the present disclosure provides a radiating system for treating a hernia of an intervertebral disc in a patient, the system comprising:
a radiating source operable to generate a laser radiation having a wavelength in the range of from 625 to 740 nm and a power in the range of from 1.5 and 150 mW; wherein
the radiating source is configured to be connected with at least one light guide for delivering the laser radiation into the patient's soft tissue, surrounding the hernia.

In a particular embodiment of the radiating system of this aspect, the power of the laser radiation generated by the radiation source is from 1.1 to 75 mW, preferably from 4 to 30 mW, more preferably from 7 to 25 mW.

In another particular embodiment of the radiating system, the wavelength of the laser radiation is from 630 to 700 nm, preferably from 630 to 680 nm, more preferably from 630 to 650 nm.

In another particular embodiment of the radiating system, the radiating system is capable of generating a laser radiation for a time from 1 to 500 min, preferably from 45 to 100 min, more preferably from 60 to 100 min.

In another particular embodiment of the radiating system, the light guide is for delivering the laser radiation into the soft tissue in the plane of the intervertebral disc being treated±0.5 cm, in the anterior direction at a distance from 0.5 to 1.5 cm away from the intervertebral joints.

In another particular embodiment of the radiating system, the light guide is for delivering the laser radiation into the soft tissue at a distance from 1.5 to 2.5 cm away from the midsagittal plane passing through the spinous processes.

In another particular embodiment of the radiating system, the soft tissue is the muscle tissue.

In another particular embodiment of the radiating system, the muscle tissue is the tissue of the dorsal paravertebral muscles.

In another particular embodiment of the radiating system, the light guide is for delivering the laser radiation into the tissue of the dorsal paravertebral muscles at a distance at least 1.0-1.5 cm away from the border of the dorsal paravertebral muscles with the thoracolumbar fascia.

In another particular embodiment of the radiating system, the radiation source is configured to be connected with more than one light guide, in particular, with from 2 to 8 light guides.

In another particular embodiment of the radiating system, the radiating system is configured for use for indirect intravascular laser blood irradiation.

In another particular embodiment of the radiating system, the use for indirect intravascular laser blood irradiation comprises subjecting a carrier solution to a laser radiation and simultaneously introducing the carrier solution being irradiated into the patient's vascular system.

In another particular embodiment of the radiating system, the use for indirect intravascular laser blood irradiation comprises subjecting a carrier solution to a laser radiation having a wavelength in the range of from 625 to 740 nm and a power in the range of from 1 to 15 mW and for a time period of from 5 to 120 min.

In another particular embodiment of the radiating system, the carrier solution comprises NaCl, nicotinate, vitamin C, and water.

In another particular embodiment of the radiating system, the radiating system is configured for use for direct intravascular laser blood irradiation.

In another particular embodiment of the radiating system, the use for direct intravascular laser blood irradiation comprises directly subjecting the patient's blood to a laser radiation having a wavelength in the range of from 625 to 740 nm and a power in the range of from 1 to 15 mW, more preferably in the range of from 1 to 7 mW, even more preferably in the range of from 1.5 to 3 mW, for a time period of from 5 to 120 min.

In another particular embodiment of the radiating system, the hernia has a size in the range of from 1 to 40 mm.

In another particular embodiment of the radiating system, the hernia of the intervertebral disc is a sequestered hernia of the intervertebral disc.

In another particular embodiment of the radiating system, the light guide is disposed inside a needle.

In another aspect, the present disclosure provides use of the radiating system according to any one of the embodiments for treating a hernia of an intervertebral disc in a patient.

The disclosed system and use allows treatment of a hernia of the intervertebral disc using low-intensity laser radiation, without directly impacting the hernia, with simultaneous plastic repair (filling) of the hernial canal with local tissues. This method allows treating an intervertebral disc hernia of any region of the vertebral column, any location, any diameter, including the sequester.

In another aspect, the present disclosure provides use of a device generating laser radiation for treating a hernia of an intervertebral disc in a patient, comprising introducing at least one light guide into the patient's soft tissue, surrounding the hernia, and subjecting the soft tissue to a laser radiation using said light guide, the laser radiation having a wavelength in the range of from 625 to 740 nm and a power in the range of from 1 and 100 mW.

In another aspect, the present disclosure provides a device generating laser radiation for implementing the method of treating a hernia of an intervertebral disc in a patient, the method comprising introducing at least one light guide into the patient's soft tissue, surrounding the hernia, and subjecting the soft tissue to a laser radiation using said light guide, the laser radiation having a wavelength in the range of from 625 to 740 nm and a power in the range of from 1 and 100 mW.

Other and further aspects and features will be evident from reading the following figures and detailed description.

### DESCRIPTION OF THE FIGURES

FIG. 1 shows an MR image of the spine of a patient (left—the cross-section in the midsagittal plane, right—the cross-section in the plane of the L5-S1 intervertebral disc). The left image shows a line that marks the plane of the L5-S1 intervertebral disc being treated. The right image shows the thickness of subcutaneous fat and the distance from the outer surface of the skin to the intervertebral joints at the level of the L5-S1 disc.

FIG. 2 shows an MR image of the spine of the patient of example 7 (a cross-section in the midsagittal plane) before treatment. The lines and crosses indicate location of the hernias (L4-L5 and L5-S1 intervertebral discs); the numbers indicate the size of the hernias.

FIG. 3 shows an MR image of the spine of the patient of example 7 (a cross-section in the in the plane of the L4-L5 intervertebral disc) before treatment. The lines and crosses at the top indicate location of the hernia; the numbers indicate the size of the hernia. The lines and crosses at the bottom indicate points of introduction of the light guides; the numbers indicate the depth of introduction of the light guides.

FIG. 4 shows an MR image of the spine of the patient of example 7 (a cross-section in the in the plane of the L5-S1 intervertebral disc) before treatment. The lines and crosses at the top indicate location of the hernia; the numbers indicate the size of the hernia. The lines and crosses at the bottom indicate points of introduction of the light guides; the numbers indicate the depth of introduction of the light guides.

FIG. 5 shows an MR image of the spine of the patient of example 7 (a cross-section in the midsagittal plane) after treatment. The lines and crosses indicate location of the hernias (L4-L5 and L5-S1 intervertebral discs); the numbers indicate the size of the hernias.

### DETAILED DESCRIPTION

The following detailed description provides means and methods using which the proposed method, systems, uses and devices can be implemented, as well as provides examples of their implementation.

When a particular embodiment is described with a reference to the method, it is understood that characteristics of this particular embodiment equally apply to systems, uses and devices according to the present disclosure, and vice versa, unless indicated otherwise or follows from the context.

Technical and scientific terms used herein have the meaning commonly understood by one skilled in the art, unless otherwise defined.

When characterizing certain quantitative features, the term “about” may be used. This term reflects uncertainty that is inherent to the measurement of any quantitative characteristic, and refers to a range that represents a quantitative characteristic±measurement error. The measurement error may be 10%, preferably 5%.

In the context of the present disclosure, the term “intervertebral disc hernia” refers to a displacement of the nucleus pulposus of the intervertebral disc, including displacements that are accompanied by a rupture of the fibrous ring.

Based on their size, intervertebral disc hernias may be divided into 4 groups:
1) small hernia (1 to 5 mm), in this case formation of protrusion of the disc is observed, in which only the inner fibers of the fibrous ring are damaged;
2) medium hernia (6 to 8 mm), in this case the protrusion further develops into a hernia with a rupture of the fibrous ring, but with a preserved longitudinal ligament, and partial exit of the hernia into the spinal canal;
3) large hernia (9 to 12 mm) is a severe form of the disease that is often accompanied by rupture of the longitudinal ligament;
4) hernia having a size of more than 12 mm is the most severe form that is typically characterized by the formation of a sequestration with the “dropping” of the disc or part of it into the spinal canal.

The hernia of the intervertebral disc can be located in any region of the spine, have different size, as well as have a sequestration.

A sequestrated disc is a condition in which a portion of the vertebral disc fragments and migrates into the spinal canal. Such condition occurs when the nucleus pulposus of a herniated disc extrudes through the annular fibers and a piece of the nucleus breaks free. The disc fragment can compress the spinal nerve root, causing pain and symptoms similar to herniated discs.

In the context of the present disclosure, the term “soft tissues” refers to the tissues that connect, support, or surround other structures and organs of the body, not being hard tissue such as bone. Soft tissues include subcutaneous fat and muscles, as well as muscle capillaries, that surround the spine and, in particular, the hernia.

In the context of the present disclosure, the term “patient” refers to a mammal, in particular, a human in need of treatment. In a particular embodiment, the age of the patient is from 12 to 90 years. The patient may have at least one herniated disc in any location and of any size. The patients may have a sequestered disc. The patient may suffer from frequent and constant back pain.

In the context of the present disclosure, the term “light guide” refers to a fiber or filament comprising a core made of a transparent material, which is surrounded by a cladding having a different refractive index. A light guide is capable of transmitting light signals through successive internal reflections. Light guides suitable in the present method are well known in the art. In an exemplary embodiment, the light guide is “TU 9444-001-17515211-98”. As an example, the light guide diameter is about 400 μm.

In the context of the present disclosure, the term “laser radiation” refers to polarized optical electromagnetic radiation having a fixed wavelength (monochromatic), a stable phase of radiation (coherent) and low divergence of the radiation pattern. The present method uses low-intensity laser radiation, in particular, the laser radiation having a wavelength in the range of from 625 to 740 nm.

According to the present disclosure, the laser radiation can be generated in pulse mode or in continuous mode.

Devices suitable for generating laser radiation according to the present disclosure, i.e. devices that can serve as a radiating source operable to generate a laser radiation are any devices providing necessary wavelength and power. Non-limiting examples of which include the following: “ALOK”, “ALOK-1”, “MATRIX-VLOK”, “UZORMED-B-2K-VLOK”, “LAZMIK”, “MULAT”, “Shuttle Combi IR+”, “AFL” and other devices having similar characteristics. A particular example is “ALOK-1” device, which is a helium neon laser.

Different type of laser generating devices can be used according to the present disclosure, e.g. gas lasers, solid state lasers, semiconductor lasers.

In an embodiment, at least one light guide is introduced into the soft tissues surrounding the hernia of intervertebral disc in the patient's body.

In a particular embodiment, the light guide is introduced inside a needle or any other device suitable for introducing a light guide into a specific location inside a patient's body. An example of such a device is a light guide attachment. When referring to introducing a light guide into a particular location inside patient's body and when a light guide attachment is used, it is understood that it is the part of the light guide attachment that emits laser radiation is introduced into the specified location inside patient's body.

The light guide may be introduced under local anesthesia.

The light guide may be introduced into the soft tissues located at a distance of up to about 5 cm from the spinous processes of the vertebra adjacent to the intervertebral disc being treated. In particular, the light guide may be introduced into the soft tissues located at a distance of from about 1 to about 4 cm, preferably from about 1.5 to about 2.5 cm from the spinous processes of the vertebra adjacent to the intervertebral disc being treated.

Because the light guide is introduced into the soft tissues located at a distance of up to about 5 cm from the spinous processes, the use of X-ray control is not required to orient the light guide or needle. In addition, because the proposed method does not require direct impact on the intervertebral disc, i.e. the light guide or the needle is not introduced into the nucleus pulposus, the method provides irradiation of the soft tissues that are in vicinity of the hernia, which is less dangerous to human health and is simple in implementation.

In yet another embodiment, the light guide is introduced to the depth of about 0.5 to about 4 cm, preferably from about 1 to about 3 cm from the skin surface.

In a particular embodiment, the light guide is introduced into the soft tissue in the plane of the intervertebral disc being treated±1.5 cm, more preferably ±1 cm, even more preferably ±0.5 cm.

In a particular embodiment, the light guide is introduced into the soft tissue at a distance from 1 to 3 cm, more preferably from 1.5 to 2.5 cm away from the midsagittal plane passing through the spinous processes.

In a particular embodiment, the light guide is introduced into the tissue of the dorsal paravertebral muscle at a distance of at least 0.5 cm away from the border of the dorsal paravertebral muscles.

On average, the depth of insertion of light guide needles into the paravertebral muscles is 1.00-2.50 cm from the own fascia of the neck and the lumbar-thoracic fascia, i.e. in the cervical, thoracic and lumbar spine. Preferably, the end of the light guide placed in the posterior paravertebral muscles is not closer than 0.50 cm to the fascia and intervertebral joints. In other words, when introducing the light guide in the soft tissues, the end of the light guide is preferably introduced at a distance of more than 0.50 cm from the fascia and stop at a distance of not less than 0.50 cm from the intervertebral joint.

In another embodiment, the location of the light guide insertion may be determined based on the thickness of the subcutaneous fat and the size of the dorsal paravertebral muscles.

In that case the rough target depth of introduction of the light guide into the soft tissue is calculated as follows: “depth=(distance from the outer surface of the skin to the intervertebral joints+depth of subcutaneous fat)/2” and the light guide shall be introduced at a depth from the skin surface in the range from “the depth of the subcutaneous fat+0.5 cm” to “the distance from the outer surface of the skin to the intervertebral joints−0.5 cm”.

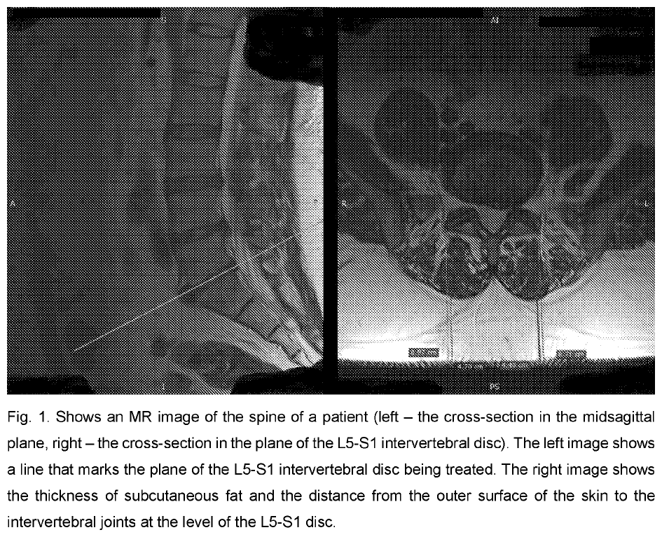

Illustration of this approach is shown on FIG. 1. Here the distance from the outer surface of the skin to the intervertebral joints is 8.97 cm (right) and 8.71 cm (left) and the depth of subcutaneous fat is 4.70 cm (right) and 4.40 cm (left). Then the target depth is

$$\begin{matrix}( 8.97 cm + 4.7 cm ) / 2 = 6.84 cm ( right ) , \\ ( 8.71 cm + 4.4 cm ) / 2 = 6.56 cm ( right ) ,\end{matrix}$$

and the depth range for introducing the light guide is:

$$\begin{matrix}from 4 , 70 + 0 , 5 = 5 , 2 cm to 8 , 97 - 0 , 5 = 8 , 47 cm ( left ) , \\ from 4 , 40 + 0 , 5 = 4 , 9 cm to 8 , 71 - 0 , 5 = 8 , 21 cm ( left ) .\end{matrix}$$

All manipulations (i.e. introduction of the light guide into the soft tissues of the patient) are performed according to anatomical landmarks, therefore, X-ray/CT/MRI/ultrasound or another type of monitoring or navigation is not required.

The disclosed method comprises subjecting the soft tissues of the patient, surrounding the hernia, to laser radiation.

The total daily dose of laser radiation for one patient can be calculated as follows:

E=WT,

where E is the dose (W·s/cm² or J/cm²), W is the intensisity (W/cm²), T is the exposure (seconds).

$$W = {P}_{av} / S ,$$

where Pav is the average power (W), S is the area of the surface being radiated.

The power can be calculated using the Beer-Lambert-Bouguer law

$$P = {P}_{0} {e}^{- kl} ,$$

where P is the final radiation power, P₀ is the initial radiation power, e is the base of the natural logarithm, k is the absorbance coefficient for this wavelength, l is the layer thickness (cm),

$$k = a + s ,$$

where α is the absorption (attenuation) coefficient, s is the loss coefficient,

s=2πrh,

where r is the radius of the vessel, h is the height of the cylinder.

In one embodiment, the power of the laser radiation is from 3 to 20 mW, preferably from 3 to 10 mW. At the same time, the power of the laser radiation of up to about 100 mW does not cause burns and may be used. In a particular embodiment, the power of the laser radiation is from 1 to 15 mW, particularly from 3 to 8 mW, more particularly from 3 to 5 mW. In yet another embodiment, the power of the laser radiation is about 5 mW, particularly about 7 mW.

Effective total dose of laser radiation of tissues per a single light guide may be about 2 J/cm², particularly about 1.5 J/cm², more particularly from 1.1 to 1.5 J/cm². As was confirmed by experimental studies on animals, laser radiation leads to increased microcirculation in the tissue. According to the studies carried out by the present inventors, allowable dose of exposure per a single light guide may be about 2 J/cm². Increased total dose of more than about 2 J/cm² may have a negative effect.

In a particular embodiment, the wavelength of the laser radiation is from 625 to 740 nm, i.e. in the red spectrum. It has been found that all wavelengths within this spectrum have similar effects on the tissue, therefore, any wavelength within this range will be useful.

The proposed method requires subjecting the soft tissues to laser radiation for a period of time, also referred to as “exposure time”.

The exposure time may be determined by a skilled practitioner based on such factors as general health conditions of the patient and the desired result.

In a particular embodiment, the exposure time varies depending on the hernia size. In another particular embodiment, the exposure time is the same for hernias having different size.

Typically, the exposure time is at least 30 minutes. In a particular embodiment, the time of subjecting the tissue to the laser radiation is from 1 to 500 min, preferably from 45 to 100 min, more preferably from 60 to 100 min. In an exemplary embodiment, the exposure time is about 60 minutes.

In some embodiments, not more than a single procedure of laser radiation per hernia is carried out per day. At the same time, if several hernias are to be treated, laser radiation of these hernias is carried out sequentially or simultaneously.

In some embodiments, from 10 to 40 procedures may be sufficient for treating hernia of any size. In a particular embodiment, the treatment is carried out for a period of time from 15 to 40 days. The time necessary for the cure may depend on the size of the hernia.

The time when the decease can be considered as cured or alleviated and when the treatment can be stopped may be determined by a skilled practitioner.

The method is preferably performed without administering medicaments to a patient. Medicaments may be optionally used when treating hernia of intervertebral disc to achieve further benefits.

In yet another embodiment more than one hernia can be treated simultaneously, in particular, from 2 to 6 hernias can be treated simultaneously. In a particular embodiment, from 2 to 6 hernias can be treated either simultaneously or sequentially, preferably from 2 to 4 hernias can be treated either simultaneously or sequentially.

The possibility of simultaneous treatment of several hernias provides a reduction of time necessary for the treatment compared with sequential treatment of the hernias.

In yet another embodiment, a single hernia is subjected to the laser radiation using more than one light guide, in particular, from 2 to 8 light guides. In a particular embodiment a single hernia is subjected to the laser radiation using from 1 to 4 light guides, for example, 2 hernias can be treated with the laser radiation using 4 light guides, in particular, using 2 light guides per each hernia. In a particular embodiment, a single hernia is subjected to the laser radiation using 4 light guides, where the two light guides are placed from one side of the hernia, or one side of the vertebral column. Thus, the method provides the possibility of treating as many as 4 hernias simultaneously or even more. In one embodiment, the light guides are introduced into the soft tissues from different sides of the hernia.

Applying several light guides to a single hernia accelerates the treatment process.

The proposed method is useful for treating hernia of any size, in particular, the hernia may have has a size in the range of from 1 to 40 mm. In a particular embodiment, the size of hernia is from 1 to 20 mm, preferably from 1 to 15 mm. In a particular embodiment, the size of hernia is less than 6 mm, preferably from 1 to 6 mm. In other particular embodiment, the size of hernia is more than 6 mm, preferably, from 6 to 11 mm. As a result of the treatment, a gradual removal of the hernia occurs.

In yet another embodiment, the hernia of intervertebral disc is a sequestered hernia of the intervertebral disc. While treating a sequestered hernia, the sequestration is gradually removed, eventually resulting in complete restoration of the ruptured fibrous ring.

In yet another embodiment, the method further comprises the step of indirect intravascular laser blood irradiation. The step of indirect intravascular laser blood irradiation is an optional procedure, however, it provides acceleration of the treatment process.

In the context of the present disclosure, the term “indirect intravascular laser blood irradiation” (indirect ILBI) refers to an invasive method of laser blood irradiation, where a light guide is introduced, for example, using a sterile needle, not directly into the vein, but into a carrier solution, which is being subjected to a laser radiation though the light guide. The irradiated carrier solution is then administered to the circulatory system, for example, through a dropper. In an embodiment, the carrier solution is prepared by combining NaCl, nicotinate, vitamin C, and water. In particular, the carrier solution may be prepared by combining 200 ml of 9% solution of NaCl, 2 ml of 1% solution of nicotinate, and 2 ml of 5% solution of vitamin C. The indirect intravascular laser blood irradiation can be performed by a nurse in the physiotherapy room.

In yet another embodiment, the step of indirect intravascular laser blood irradiation is carried out with a wavelength in the range of from 625 to 740 nm and a power in the range of from 1 to 15 mW during a time period of from 5 to 120 min. In a particular embodiment, the wavelength in the range of from 630 to 700 nm, preferably from 630 to 680 nm, more preferably from 630 to 650 nm, more preferably 635 nm. In a particular embodiment, the power of the laser radiation in the range of from 3 to 15 mW, preferably from 6 to 15 mW.

In yet another embodiment, the method further comprises the step of direct intravascular laser blood irradiation. The direct intravascular laser blood irradiation is optional, however, in case it is carried out it provides acceleration of the treatment process.

In the context of the present disclosure, the term “direct intravascular laser blood irradiation” (direct ILBI) refers to invasive method of laser blood irradiation that requires a contact of light with blood. In this case, a light guide is introduced directly into the vascular system, for example, a light guide is inserted into the vein through an injection needle or a catheter. The other end of the light guide is connected to the laser therapy system (device). Thus, blood in the vein, while passing near the distal end of the light-guide, is exposed to the laser radiation. The laser radiation is absorbed by different blood components, including the erythrocytes, the blood platelets, the leukocytes, the lymphocytes, and the blood proteins. Helium-neon (HeNe laser) (632.8 nm) or semiconductor laser diodes (633, 635 nm) may be used to perform the intravascular laser blood irradiation.

In yet another embodiment, the laser radiation is carried out with a laser radiation having a wavelength in the range of from 625 to 740 nm and a power in the range of from 1 to 15 mW during a time period of from 5 to 120 min.

In yet another embodiment, the direct intravascular laser blood irradiation is carried out with a laser radiation having a wavelength in the range of from 625 to 740 nm and a power in the range of from 1 to 15 mW during and a time period of from 5 to 120 min. In a particular embodiment, the laser radiation is carried out with a laser radiation having a wavelength in the range of from 630 to 700 nm, preferably from 630 to 680 nm, more preferably from 630 to 650 nm, more preferably 630 nm. In a particular embodiment, the laser radiation is carried out with a laser radiation having a power in the range of from 1 to 7 mW, preferably from 1.5 to 3 mW.

The intravascular laser blood irradiation is performed on an outpatient basis.

Further advantages of intravascular laser blood irradiation are its simplicity in implementing and comparative accessibility. Compared to drugs, laser radiation does not cause allergic reactions and is well tolerated by patients, with the exception of rare cases of increased photosensitivity.

Without wishing to be bound by a particular theory, the present inventors believe that biological effects of the laser radiation are based on its anti-inflammatory, reparative, immunostimulatory, and bacteriostatic action that enhances the regenerative processes.

Advantages of the proposed method include the following:
the method neither requires cuts nor leads to injuries;
the method allows complete regeneration of all damaged elements of the disc with the patient's own cells;
the method allows treating (removing) multiple hernias;
the method does not lead to complications or relapses;
the method provides full recovery of the spinal function;
the method provides pain relief on the 1-15 day of the treatment;
the method does not require overnight stay in a hospital;
the method does not require rehabilitation.

Further advantage of the proposed method is that the laser beam does not damage the normal tissues, therefore, regeneration is possible when treating a hernia. The present inventors note that in patients treated by the proposed method, both the fibrous ring and the nucleus pulposus were restored. In surgical aspect, this phenomenon should be considered as plastic surgery with local tissues.

The proposed method provides the possibility of non-invasive treatment of an intervertebral disc hernia until its complete disappearance and without relapse of the disease. Further beneficial effect of the method is the possibility of outpatient treatment, absence of cicatricial changes, absence of postoperative complications, and low probability of recurrence of the disc hernia. The method also provides treatment without damaging essential structures such as nerve endings, blood vessels, etc. In addition, along with the treating the hernia, so-called hernia canal plastic surgery with local tissues is performed, which in the future excludes hernia recurrence in this segment.

The list of areas in which the proposed invention can be used includes, but is not limited to the following (according to the classification of diseases according to ICD-10):
M50.0+ Cervical disc disease with myelopathy (G99.2*);
M50.1 Cervical intervertebral disc disease with radiculopathy;
M50.3 Other cervical intervertebral disc degeneration;
M50.8 Other lesions of cervical intervertebral disc;
M50.9 Disorder of cervical intervertebral disc, unspecified;
−M51.0+ Disorders of the intervertebral discs of the lumbar and other parts with myelopathy (G99.2*);
M51.1 Disorders of lumbar and other intervertebral discs with radiculopathy;
M51.3 Other specified intervertebral disc degeneration;
M51.8 Other specified lesion of intervertebral disc;
M51.9 Disorder of intervertebral disc, unspecified.

In another aspect, the present disclosure relates to a radiating system for treating a hernia of an intervertebral disc in a patient. This radiating system may be used for implementing the method disclosed herein.

The radiating system comprises a radiating source operable to generate a laser radiation having a wavelength in the range of from 625 to 740 nm.

The system may have more than one radiating source, power of each radiating source may be adjusted independently from one another, but in some embodiments may be the same.

In some embodiments, the radiating source or sources may be configured to be connected to a common control unit that controls power of the radiating source, time of its operation, and mode of its operation.

In some embodiments, each radiating source has its own control unit that controls power of the radiating source, time of its operation, and mode of its operation.

In a particular embodiment, the radiating system comprises one or more control units.

The power of the radiating source, time of its operation, and mode of its operation may be set by the user in the control unit or units.

The radiating source operable to generate a laser radiation may be based on different types lasers, e.g. gas lasers, solid state lasers, semiconductor lasers.

The radiating source may generate laser radiation in a continuous or a pulse mode. The pulse mode may be characterized by a frequency of pulses. In some embodiments, the frequency of of pulses may be in the range of from 0.1 to 100 Hz.

The method disclosed herein is effective for all modes of operation of the radiating source.

The radiating source may configured to be connected with at least one light guide for delivering the laser radiation into the patient's soft tissue, surrounding the hernia.

In a particular embodiment, the radiating system comprises one or more light guides.

A power of the radiating source may be in the range of from 1.5 to 150 mW. If the radiating system has more than one radiating source, each radiating source has the power in the range of from 1.5 and 150 mW. If several light guides are connected to a single radiating source, the radiating source has a power in the range of from 1.5 and 150 mW per one light guide.

The light guide may be configured to be connected with a light guide attachment, for example, comprising a needle for ease of introduction into the patient's soft tissue, surrounding the hernia. An example of such an attachment is KIVL-01.

In a particular embodiment, the radiating system comprises one or more light guide attachments.

One skilled in the art appreciates that the laser radiation when passing from the radiating source to the patient's soft tissue though a laser guide and a laser guide attachment loses its power.

Therefore, in some embodiments the power of the radiating source is such that the power of laser radiation delivered into the patient's soft tissue, surrounding the hernia, is in the range of from 1 to 100 mW. If the radiating system has more than one radiating source, each radiating source provides delivery of laser radiation with a power in the range of from 1 to 100 mW. If several light guides are connected to a single radiating source, the radiating source provides delivery of laser radiation with a power in the range of from 1 to 100 mW per one light guide.

In some embodiments, the radiating system has means for controlling the power delivered into the patient's soft tissue, for example, at the exit of a light guide attachment.

Other and further aspects and features will be evident from reading the following examples.

### EXAMPLES

The following examples further describe and demonstrate particular embodiments within the scope of the present disclosure. It is to be understood that the examples are provided to illustrate certain embodiments and that no limitation to the scope is intended thereby.

The studies were carried out on a group of people having hernias in different parts of the spine.

Before starting the treatment, MRI of the spine, a blood test, and electrocardiogram were done for each patient.

### General Method of Treatment

The operating field was preliminary treated three times with antiseptic solutions. A needle with a light guide inside was introduced into the soft tissues, in particular, into the zone of the dorsal paravertebral muscles, of a patient being under local anesthesia (using 1-2 ml of SOL lidocaine 2%). The patient in the prone position for the lumbar and thoracic spine, in the sitting position for the cervical spine. The needle was introduced at an angle of 80 degrees and a distance of 1.0-3.0 cm from the spinous processes of the spine perpendicular to the midline of the spine of certain segments, and to a depth of 1.0-3.0 cm from the lumbar-thoracic fascia at the level of the herniated disc. In order to determine the depth of introduction of light guide needles, for each patient the thickness of the subcutaneous fat was assessed according to the results of MRI, followed by an individual selection of needles, as well as the depth of their introduction. After introducing the needles into the soft tissue, the needles are fixed and connected to a laser device (such as “ALOK-1”, “MATRIX-VLOK”, “UZORMED-B-2K-VLOK”, “Shuttle Combi IR+”, “LAZMIK”, or other devices with similar characteristics) having a wavelength of 0.63 microns and output power of 1 to 5 mW. The duration of the procedure was selected individually in each case. The total dose of laser radiation was not more than 1.5 J/cm² (the calculation was carried out according to the formula described in detail above). Manipulations were done to 1-6 hernias simultaneously. The procedures were carried out daily 5 days a week for 10-50 days.

### The Procedure of Indirect Intravenous Laser Blood Irradiation (Indirect ILBI)

The patients were subjected to indirect intravenous laser blood irradiation (indirect ILBI) using devices such as ALOK-1, “Matrix VLOK”, “UZORMED-B-2K-VLOK”, “Shuttle Combi IR+”, or “LAZMIK” with a wavelength of 0.63 μm and output power of 1-5 mW. Losses in a collimator and the light guide were about 50%; the output power of the expendable guide was about 0.5-2.5 mW. During the procedure, a sterile light guide was introduced not directly into the vein, but into the carrier solution, which was administered to a cubital vein through a dropper. The solution consisted of 200 ml of NaCl 0.9%, 2 ml of nicotinate 1%, 2 ml of ascorbic acid (vitamin C) 5%. The solution itself was a light guide, but one which significantly suppressed the radiation. The time of a single exposure was 60-90 minutes. The radiation dose was 1.2 J/cm². The indirect ILBI was carried out once in 7 days, the procedure was repeated 5 times.

### The Procedure of Direct Intravenous Laser Blood Irradiation (Direct ILBI)

The patients were subjected to direct intravenous laser blood irradiation (direct ILBI) using devices such as ALOK-1, “Matrix VLOK”, “UZORMED-B-2K-VLOK”, “LAZMIK”, “Shuttle Combi IR+” with a wavelength of 0.63 microns and output power of 1-5 mW. Losses in the collimator and the guide were about 50%; the output power of the expendable guide was about 0.5-2.5 mW. During the procedure, a sterile guide was introduced directly into the vein. Time of a single exposure was 30 minutes. The radiation dose was 1.2 J/cm². Direct intravenous laser blood irradiation was carried out once in 7 days, the procedure was repeated 5 times.

### Example 1

### Male Patient of the Age of 45

### Before the Treatment, the Patient's Health Condition was the Following.

On a series of MRIs in T1, T2-weighted imaging in standard planes, lumbar lordosis was smoothed.

Degenerative changes of intervertebral disc were determined, the height of the L4-S1 discs was reduced. At the level of L4-L5, associated with dorsal diffuse protrusion of the intervertebral disc, a left-sided foraminal hernia of up to 4.5 mm in size was visualized, it narrowed the intervertebral opening, distal parts of the root without signs of edema.

At the L5-S1 level, associated with the dorsal diffuse protrusion, there was a fragment of the fibrous ring rupture with formation of a sequestered median-cranial distribution along the midline up to 7-8 mm (connection with the intervertebral disc was preserved). The hernia was characterized by an iso-intensive MR signal at T1-weighted imaging, caused deformation of an anterior wall of the dural sac, lateral portions of side pockets, narrowed the intervertebral foramen with compression of the corresponding spinal roots, distal roots without signs of edema, anterior epidural tissue with signs of slight edema. Median sagittal size of the spinal canal was: at the L1-L2 level-up to 22 mm, L2-L3-19 mm, L3-L4-20 mm, L4-L5-19 mm, L5-S1-5-6 mm.

The posterior and anterior longitudinal ligaments were unevenly thickened at the level of the intervertebral discs. Along adjacent surfaces of the vertebral bodies, marginal bone points were visualized.

The height of the vertebral bodies was preserved. The intensity of the MR signal from the bone marrow of the vertebral bodies on T1- and T2-weighted imaging was not changed, there were small areas of the bone marrow edema along adjacent surfaces of the vertebral bodies L5, S1.

Degenerative changes of the arched joints were determined in the form of an uneven decrease in the height of the articular cartilage, subchondral sclerosis. The articular facets and the yellow ligaments at the level of L4-L5, L5-S1 were unevenly hypertrophied.

The contours of the cone of the spinal cord and cauda equina were clear, even, their MR structure was not changed.

Thus, the patient had osteochondrosis, initial spondylarthrosis, herniated discs L4-L5, L5-S1, as well as narrowed spinal canal and side pockets at L5-S1 level, where the sequestrated hernia of L5-S1 had the size of 13.5 mm.

The patient was treated with low intensity laser radiation as follows. Two 0.38×0.8 mm needles with a light guide inside (TU 9444-001-17515211-98) were introduced into the soft tissues of a patient being under local anesthesia (using 1-2 ml of SOL lidocaine 2%), one needle from the left side and the other from the right side of L5-S1. The needles were introduced at an angle of 80 degrees and a distance of about 2 cm from the spinous processes of the spine perpendicular to the midline of the spine of certain segments, to a vertical depth of about 3 cm. The light guide was connected to the laser device “UZORMED-B-2K-VLOK” having a wavelength of 0.63 microns. The exposure time was 90 minutes per each needle, with a total dose of laser radiation about 1.5 J/cm². 31 procedures were carried out within 6 weeks.

In addition, the patient was further treated with indirect ILBI using an aqueous carrier solution comprising NaCl, Vitamin C, Nicotinate using “UZORMED-B-2K-VLOK” with a wavelength of 0.63 μm. Indirect ILBI was carried out once in 7 days, the procedure was repeated 5 times. The exposure time was 60 minutes.

After the treatment, MRI showed the following:

Lumbar lordosis was smoothed.

Signs of a degenerative-dystrophic process were determined in the form of:
decrease in signal intensity from the intervertebral discs due to dehydration of the pulp nuclei, the most significant changes at the level L4-L5, L5-S1;
small marginal anterior-lateral bone growths along adjacent sections of the L5-S1 vertebral bodies;
moderate narrowing of the joint spaces, induration of the articular areas of the arched joints.

Left-sided foraminal hernia of the intervertebral disc L4-L5 was determined having a size of up to 4.1 mm, against the dorsal protrusion, deforming the anterior subarachnoid space, with narrowing the intervertebral openings (D<S), without affecting the roots. Sagittal lumen of the spinal canal was 17 mm.

Medial protrusion of the intervertebral disc L5-S1 of up to 2.8 mm was determined, deforming anterior subarachnoid space, with narrowing intervertebral openings, without affecting the roots. Sagittal lumen of the spinal canal was 15 mm.

Pulposus nuclei of the intervertebral discs of the spine were located within fibrous capsules and did not prolapse into lumen of the spinal canal.

Configuration and structure of the cone of the spinal cord, elements of cauda equina were not changed. MRI signs of circulatory disorders were not detected. The paravertebral soft tissue was not changed.

Conclusion: MR demonstrates a moderate osteochondrosis with statics failure, moderate signs of spondylarthrosis; L4-L5 disc hernia, L5-S1 disc protrusion, where the protrusion of L5-S1 was less than 2.8 mm. In comparison to the examination before the treatment positive dynamics was observed.

TABLE 1
The patient's condition before and after the treatment

| Patient of | | |
|---|---|---|
| e×ample 1 | Before the treatment | After the treatment |
| Diagnosis | sequestrated hernia | protrusion of L5-S1 |
| | of L5-S1 having a size | of less than 2.8 mm |
| | of 13.5 mm | |

### Example 2

### Male Patient of the Age of 36

### Before the Treatment:

On MRI tomograms, in T2-weighted imaging, T1-weighted imaging, and STIR modes in the sagittal, coronal and axial planes, the structures of the lumbar spine were visualized.

The physiological lumbar lordosis was smoothed.

Signs of degenerative-dystrophic changes in the intervertebral discs were determined, characterized by a hypo-intense MR signal from T2-IP from pulposus nuclei and a moderate decrease in the height of the discs.

In L4-L5 segment, a median hernia of the intervertebral disc having a size of up to 4 mm was determined, displacing back the transient roots. The intervertebral foramen were narrowed, the outgoing roots were not compressed.

The anteroposterior size of the dural space at this level was 1 cm.

In segment L5-S1, a paramedian left-sided hernia of the intervertebral disc was visualized having a size of up to 5 mm, with signs of caudal migration with formation of a sequestration having a size of 11*10 mm with compression of the left root.

The intervertebral foramen were narrowed, the outgoing roots were not compressed.

The anteroposterior size of dural space at this level was 1.2 cm.

The vertebral bodies have usual shape and size, and MR signal from the bone marrow was not changed.

The intervertebral joints were characterized by signs of initial spondylarthrosis in the form of narrowing of the joint spaces, thinning of the articular cartilage, and hypertrophy of the articular facets.

The spinal cord at the scanning level and the cauda equina were not enlarged, had clear contours and a homogeneous structure, the intensity of the MR signal was not changed. The cerebrospinal fluid circulation was intact. The paravertebral soft tissues were not changed.

Conclusion: MRI showed signs of degenerative-dystrophic changes in the lumbar spine in the form of spondylosis and spondylarthrosis, and intervertebral disc hernias L4-L5, L5-S1, where L5-S1 hernia was a sequestrated hernia having a size of 11 mm.

The patient was treated with low intensity laser radiation as follows. Two 0.38×0.8 mm needles with a light guide inside (TU 9444-001-17515211-98) were introduced into the soft tissues of a patient being under local anesthesia (using 1-2 ml of SOL lidocaine 2%), one needle from the left side and the other from the right side of L5-S1. The needles were introduced at an angle of 80 degrees and a distance of about 2 cm from the spinous processes of the spine perpendicular to the midline of the spine of certain segments, to a vertical depth of about 3 cm. The light guide was connected to the laser device “Shuttle Combi IR+” having a wavelength of 0.635 microns. The exposure time was 90 minutes per each needle, with a total dose of laser radiation about 1.5 J/cm². 30 procedures were carried out within 3 months.

In addition, the patient was further treated with indirect ILBI using an aqueous carrier solution comprising NaCl, Vitamin C, Nicotinate using “Shuttle Combi IR+” with a wavelength of 0.635 μm. Indirect ILBI was carried out once in 7 days, the procedure was repeated 5 times. The exposure time was 60 minutes.

After the treatment, MRI showed the following:

In T2-weighted imaging, T1-weighted imaging, STIR modes in the sagittal, coronal and axial planes, structures of the lumbar spine were visualized.

The physiological lumbar lordosis was smoothed.

Signs of degenerative-dystrophic changes in the intervertebral discs were observed, characterized by a hypo-intense MR signal from T2-IP from the pulposus nuclei and a moderate decrease in the height of the discs.

In segment L4-L5, a median hernia of the intervertebral disc was observed having a size of up to 5 mm, pushing back the transient roots. The intervertebral foramen were narrowed, the outgoing roots were not compressed.

The anteroposterior size of the dural space at this level was 1.5 cm.

In segment L5-S1, a paramedian left-sided hernia of the intervertebral disc was visualized having a size of up to 6 mm, having signs of compression of the left root. The intervertebral foramen were narrowed, the outgoing roots were not compressed.

The anteroposterior size of the dural space at this level was 1.4 cm.

The vertebral bodies had usual shape and size, the MR signal from the bone marrow was not changed.

The intervertebral joints had signs of initial spondylarthrosis in the form of narrowing of the joint spaces, thinning of the articular cartilage, and hypertrophy of the articular facets.

The spinal cord at the scanning level and the cauda equina were not enlarged, had clear contours and a homogeneous structure, intensity of the MR signal was not changed. Cerebrospinal fluid circulation was intact.

The paravertebral soft tissues were not changed.

Conclusion: MRI showed signs of degenerative-dystrophic changes in the lumbar spine in the form of spondylosis and spondylarthrosis and intervertebral disc hernias L4-L5, L5-S1, where L5-S1 hernia was 5 mm in size, the sequester was absent.

TABLE 2
The patient's condition before and after the treatment

| Patient of | | |
|---|---|---|
| e×ample 2 | Before the treatment | After the treatment |
| Diagnosis | dorsal sequestrated hernia of | hernia of L5-S1 of 5 mm, |
| | L5-S1 disc of 10*11 mm | sequester is absent |
| | radiculopathy of the left roots L5 | regression of radicular pain |
| | and S1 | syndrome |
| Symptoms | lumbar pain (recurrent, wavy during | pain syndrome completely |
| | the day), especially after lifting | disappeared; |
| | weights; | complete absence of discomfort in |
| | increased pain in the evening and at | the lower back (at any time of the |
| | night; | day); |
| | frequent insomnia due to discomfort | healthy sound sleep; |
| | in the lumbar region; | tension and feeling of fatigue in the |
| | tension of the muscles of the | muscles of the legs are absent; |
| | posterior surface of the thigh, which | increased efficiency and general |
| | over time was replaced by their | activity. |
| | hypotension (weakness); | |
| | rapid fatigability when standing and | |
| | walking. | |

### Example 3

### Male Patient of the Age of 63

### Before the Treatment:

On a series of T1 and T2-weighted MR images in the sagittal, coronal and axial planes, the lumbar lordosis was exacerbated. The left-sided scoliotic deformation of the lumbar part of the spine was determined. L4 vertebral body was displaced anteriorly by 3.5 mm in relation to the L5 vertebral body.

Hydrophilicity of the nucleus pulposus of the intervertebral discs was reduced. The height of the intervertebral discs was unevenly reduced.

Biforamal protrusion of the intervertebral disc Th12-L1 of up to 2.5 mm in size, partially narrowing the intervertebral foramen without radicular action, was determined. Sagittal size of the spinal canal was 21 mm.

At L1-L2 level, a biparaforaminal disc hernia was determined, having a size of up to 3.5 mm, deforming the anterior wall of the dural sac, partially narrowing the intervertebral foramen without convincing signs of impact on the roots. Sagittal size of the spinal canal was 21 mm.

At L2-L3 level, associated with diffuse disc protrusion, a biphoraminal disc herniation was determined (more to the right), up to 4.5 mm in size, deforming the anterior wall of the dural sac, narrowing the intervertebral foramen (more the right), partially compressing the right spinal root, adjacent to left spinal root. Sagittal size of the spinal canal was 21 mm.

Levels of L3-L4 associated with diffuse protrusion of the disc, a biphoraminal disc herniation, up to 4.5 mm in size was determined, that deformed the anterior wall of the dural sac, narrowed the intervertebral foramen, partially compressed the spinal roots. Sagittal size of the spinal canal was 21 mm.

Associated with diffuse protrusion of L4-L5 intervertebral disc, a left-sided posterolateral disc herniation was determined with a sequestered part of the disc (preserving connection with the disc), which had an oval shape, which was located paraforaminally and foraminally on the left, and had a size of 0.9×1.1×1.6 cm. Subarachnoid space at this level was compressed, the intervertebral foramen were narrowed, the spinal roots were partially compressed (more on the left). Sagittal size of the spinal canal was 14 mm.

At L5-S1 level, there was a diffuse protrusion of the intervertebral disc, of up to 1.7 mm in size, partially narrowing the intervertebral foramen (more towards the right) without radicular impact. Sagittal size of the spinal canal was 14 mm.

Shape and size of the bodies of the lumbar vertebrae were unremarkable, the intensity of the MR signal from them was increased by T1-WI and T2-WI due to the zones of fatty degeneration of the bone marrow.

Marginal bone cusps were visualized along adjacent endplates on the anterior and posterior-lateral surfaces of the bodies of the lumbar vertebrae.

Integrity of the endplates of the body of Th12 vertebrae, of adjacent endplates of bodies L2-L3, cranial endplate of L4 vertebral body was impaired with formation of small Schmorl hernias without signs of perifocal edema.

Posterior longitudinal ligament was unevenly compacted.

There were signs of spondyloarthrosis of the facet joints in the form of subchondral sclerosis, uneven narrowing of the joint spaces, bone cusps, and uneven compaction of the yellow ligaments.

The spinal cord in the scanned area had a homogeneous structure, the intensity of the MR signal from it was not changed. Spinal cone was visualized at the level of L1 vertebral body.

Conclusion: MR-image of degenerative-dystrophic changes in the thoracic spine (intervertebral osteochondrosis, spondylosis, spondyloarthrosis) with impaired statics. Antespondylolisthesis at L4-L5 level. Herniated discs L1-L2, L2-L3, L3-L4, sequestered herniated disc L4-L5 of up to 9*11*16 mm. Protruded discs Th12-L1, L5-S1. Schmorl's hernias of bodies Th12, L2, L3, L4 of the vertebrae.

The patient was treated with low intensity laser radiation as follows. Two 0.38×0.8 mm needles with a light guide inside (TU 9444-001-17515211-98) were introduced into the soft tissues of a patient being under local anesthesia (using 1-2 ml of SOL lidocaine 2%), where the needles were introduced from the right side of L4-L5. The needles were introduced at an angle of 80 degrees and a distance of about 2 cm from the spinous processes of the spine perpendicular to the midline of the spine of certain segments, to a vertical depth of about 3 cm. The light guide was connected to the laser device “Shuttle Combi IR+” having a wavelength of 0.635 microns. The exposure time was 90 minutes one needle, and 60 minutes for the other needle, with a total dose of laser radiation of about 1.5 J/cm². 30 procedures were carried out within 6 weeks.

In addition, the patient was further treated with indirect ILBI using an aqueous carrier solution comprising NaCl, Vitamin C, Nicotinate, using “Shuttle Combi IR+” with a wavelength of 0.635 μm. Indirect ILBI was carried out once in 7 days, the procedure was repeated 5 times. The exposure time was 60 minutes.

After the treatment, MRI showed the following:

A series of MR-images of the lumbosacral spine in the axial, sagittal and coronal planes were obtained. Left-sided scoliosis.

Signs of degenerative-dystrophic changes were determined in the form of:
a decrease in intensity of MR signal on T2-WI from the intervertebral discs at L1-S1 level with a decrease in their heights;
anterior and posterolateral marginal bone growths of the vertebral bodies;
small central Schmorl nodes in Th12, L2-L4;
hypertrophy of yellow ligaments in L4-L5 segment;
areas of fatty degeneration of bone marrow in the vertebral bodies.

Body L4 was displaced anteriorly by 4 mm, body L5 was displaced posteriorly by 2.5 mm.

At L1-L2 level, a 2 mm median herniated disc was determined, which deformed the anterior wall of the dural sac. The intervertebral foramen were not narrowed. Anteroposterior dimension of the spinal canal at this level was 19 mm.

At L2-L3 level, associated with diffuse protrusion and posterolateral marginal bone growths, it was determined a presence of a biphoraminal herniated disc, 4 mm in size, narrowing the intervertebral foramen. Anterior wall of the dural sac was moderately deformed at this level. Anteroposterior dimension of the spinal canal at this level was 21 mm.

At L3-L4 level, associated with diffuse protrusion and posterolateral marginal bone growths, it was determined a presence of a biphoraminal herniated disc, 4 mm in size, narrowing the intervertebral foramen. The anteroposterior dimension of the spinal canal at this level was 21 mm.

At L4-L5 level, associated with antelisthesis of L4 body, diffuse protrusion of the intervertebral disc was determined, corresponding to the degree of listosis of L4 body. The intervertebral foramen were narrowed, more on the left, left L4 root was compressed in the intervertebral foramen. Anteroposterior dimension of the spinal canal at this level was 14 mm.

At L5-S1 level, a diffuse protrusion of the intervertebral disc was determined, less than 2 mm in size. The right intervertebral foramen was narrowed, mainly due to bony growths in the right intervertebral joint. Anteroposterior dimension of the spinal canal at this level was 18 mm.

Spondyloarthrosis was much more pronounced in L4-L5 segment, where there was a deformation of the joints and large bone growths.

In the body of L4, a hemangioma of 13 mm cannot be excluded.

The spinal cord and the cauda equina roots were characterized by homogeneous structure at the studied level; their MR signal was not changed.

Conclusion: MRI signs of osteochondrosis of the lumbosacral spine. Herniated discs at the level of segments L1-L2, L2-L3, L3-L4. Disc protrusion at the level of segments L4-L5, L5-S1. Antespondylolisthesis L4, retrospondylolisthesis L5. Sponylosis. Spondyloarthrosis. Scoliosis.

In comparison to the examination before the treatment, there was a regression of the paraarticular synovial cyst at the level of L4-L5 segment on the left.

TABLE 3
The patient's condition before and after the treatment

| Patient of | | |
|---|---|---|
| e×ample 3 | Before the treatment | After the treatment |
| Diagnosis | sequestrated hernia of L4-L5 | diffuse protrusion of L4-L5 |
| | having a size of 9*11*16 mm | of less than 2 mm in size |

### Example 4

### Male Patient of the Age of 45

### Before the Treatment:

The physiological lordosis was smoothed. Retrolisthesis of L5 vertebra had the size of up to 5 mm. Signs of instability in L4-5 segment. Narrow spinal canal: its anteroposterior dimension at the level of L1 vertebra was 17 mm.

The examination showed degenerative-dystrophic changes with a decrease in the height of the discs and the intensity of the MR signal from them in L2-3 and L4-S1 segments. There were multiple central Schmorl nodes in the vertebral bodies at the studied level.

Posterior-right protrusion of L2-3 disc having a size of up to 2-3 mm, deforming the anterior wall of the dural sac on the right, without root compression.

Right-sided paramedial sequestered disc herniation L4-5 was up to 7 mm in size and was associated with an extensive protrusion of the disc, compressing the dural sac and the right root. There was a caudal migration of the sequestrum to the upper ⅓ part of L5 vertebral body (sequestration length was about 12 mm). The left intervertebral foramen was also stenotic at this level.

Right-sided paramedial disc herniation L5-S1 was up to 7 mm in size and was associated with L5 vertebra listosis, with the presence of an intraforaminal right-sided component, deforming the dural sac, stenosing the right intervertebral foramen and compressing the right root.

Dorsal disc herniation with compression of the dural sac and roots at other levels was not revealed.

Arthrosis and hypertrophy of facet joints in L3-S1 segments were determined.

Conclusion: Osteochondrosis of the lumbosacral spine with impaired statics. Retrolisthesis of L5 vertebra. Sequestrated hernia of disc L4-L5 having a size of up to 7 mm (sequester 12 mm) and hernia of disc L5-S1 having a size of up to 7 mm. Disc protrusion L2-3. Spondyloarthrosis in L3-S1 segments. Narrow spinal canal.

The patient was treated with low intensity laser radiation as follows. Four 0.38×0.8 mm needles with a light guide inside (TU 9444-001-17515211-98) were introduced into the soft tissues of a patient being under local anesthesia (using 1-2 ml of SOL lidocaine 2%), one needle from the left side and the other from the right side of L5-S1 and one needle from the left side and the other from the right side of L4-L5. The needles were introduced at an angle of 80 degrees and a distance of about 2 cm from the spinous processes of the spine perpendicular to the midline of the spine of certain segments, to a vertical depth of about 3 cm. The light guide was connected to the laser device “Shuttle Combi IR+” having a wavelength of 0.635 microns. The exposure time for L4-L5 disc was 90 minutes for each needle, and exposure time for L5-S1 disc was 60 minutes for each needle, with a total dose of laser radiation about 1.5 J/cm². 35 procedures were carried out within 7 weeks.

In addition, the patient was further treated with indirect ILBI using an aqueous carrier solution comprising NaCl, Vitamin C, Nicotinate, using “Shuttle Combi IR+” with a wavelength of 0.635 μm. Indirect ILBI was carried out 10 times. The exposure time was 30 minutes.

After the treatment, MRI showed the following:

On a series of MRI, weighted by T1 and T2, including STIR, in the sagittal, coronal and axial planes, the physiological lumbar lordosis was smoothed.

Decrease in the height of the discs and intensity of MR signal from it on T2-WI was determined, especially at L3-L4, L4-L5 levels.

The following was determined:
diffuse dorsal protrusions of L1-L2, L2-L3, L3-L4 discs of up to 2 mm, deforming the anterior subarachnondal space, intervertebral foramen, without root compression;
dorsal diffuse hernia of L4-L5 disc of up to 4.5 mm in size, covered with lateral osteophytes, deforming anterior wall of the dural sac, intervertebral foramen, minimally adhering to the roots of L4 at this level;
dorsal diffuse hernia of L5-L1 disc of up to 4 mm in size, deforming the intervertebral foramen, anterior wall of the dural sac, minimally adjacent to the roots of L5 at this level.

Schmorl's hernias of bodies L1-L4 of the vertebrae were observed. Shape and size of the bodies of the remaining vertebrae were normal. MR signal from the bone marrow of the vertebral bodies was changed due to small areas of fatty degeneration. There were signs of spondyloarthrosis of the intervertebral joints at L3-S1 level.

Distal spinal cord was structural, intensity of the MR signal from it was not changed. The cone of the spinal cord was at the normal level.

Paravotebral soft tissues were within normal.

Conclusion: MR-image showed degeneration-dystrophic changes of the lumbosacral spine. Spondyloarthrosis. Osteochondrosis. Diffuse dorsal protrusions of L1-L2, L3-L3, L3-L4 discs without root compression. Hernia of L4-L5 disc had a size of less than 4.5 mm. Hernia of L5-S1 disc was less than 4 mm in size.

TABLE 4
The patient's condition before and after the treatment

| Patient of | | |
|---|---|---|
| e×ample 4 | Before the treatment | After the treatment |
| Diagnosis | sequestrated hernia of | hernia of L4-L5 less |
| | L4-L5 having a size of up | than 4.5 mm |
| | to 7 mm (sequester 12 mm) | |
| | hernia of L5-S1 disc | hernia of L4-L5 having |
| | having a size of up to 7 mm | a size of 4 mm |

### Example 5

### Male Patient of the Age of 51

### Before the Treatment:

A series of MRI shows image of lumbosacral spine (T1-WI, T2-WI, and IS STIR (impulse sequence Short T1 Inversion Recovery)) in sagittal, coronal and axial projections showed that the lumbar lordosis was smoothed. Mild left-sided scoliosis was determined.

The height of the intervertebral discs was not reduced, L4-S1 discs were dehydrated. Left-sided reframinal sequestered disc herniation L4-5 of up to 11 mm in size was determined, compressing the left root of the spinal nerve, dural sac and cauda equina fibers. Right-sided paramedial disc herniation L5-S1 with cranial migration of up to 4 mm in size, compressing the dural sac was determined.

The height of the vertebral bodies was not reduced. MR signal from the bone marrow was heterogeneous due to isolated areas of fatty degeneration. Marginal bone growths along the posterior surfaces of L3-L5 vertebral bodies were visualized, deforming the spinal canal and compressing the dural sac.

MR signal from the structures of the spinal cord was not changed.

Conclusion: Degenerative-dystrophic changes in the lumbosacral spine. Statics of the spine was impaired. Sequestrated hernia L4-L5 having a size of up to 11 mm.

The patient was treated with low intensity laser radiation as follows. Two 0.38×0.8 mm needles with a light guide inside (TU 9444-001-17515211-98) were introduced into the soft tissues of a patient being under local anesthesia (using 1-2 ml of SOL lidocaine 2%), where the needles were introduced from the right and the left side of L4-L5. The needles were introduced at an angle of 80 degrees and a distance of about 2 cm from the spinous processes of the spine perpendicular to the midline of the spine of certain segments, to a vertical depth of about 3 cm. The light guides were connected to the laser device “Shuttle Combi IR+” having a wavelength of 0.635 microns. The exposure time was 60 minutes for each needle, with a total dose of laser radiation about 1.5 J/cm². 20 procedures were carried out within 5 weeks.

In addition, the patient was further treated with indirect ILBI using an aqueous carrier solution comprising NaCl, Vitamin C, Nicotinate using “Shuttle Combi IR+” with a wavelength of 0.635 μm. Indirect ILBI was carried out once a week, 5 times in total. The exposure time was 60 minutes.

After the treatment, MRI showed the following:

On a series of MRI of the cervical spine, T1- and T2-weighted in two projections, lordosis was straightened. The height of the C5/C6 intervertebral disc was reduced, the other discs of the studied area were the same, signals from the cervical discs according to T2 were reduced. The posterior longitudinal ligament was indurated.

The ratios in the lateral atlanto-axial joints were not disturbed. The uncovertebral joints were partially acercous.

A dorsal diffuse hernia of C5/C6 disc, 0.3 cm in size, extending into the intervertebral foramen on both sides, deforming the adjacent part of the dural sac was determined.

A dorsal diffuse protrusion of C6/C7 disc, 0.2 cm in size, extending into intervertebral foramen on both sides was determined.

In the body of C7 vertebra, a section of pathological MR signal of hyperintense T2 and T1, 0.5×0.5 cm in size, of a reticular structure, probably hemangioma, was determined.

Lumen of the spinal canal was narrowed at the level of hernia and protrusion, structural signal from the spinal cord (according to T1 and T2) was not changed. The shape and the size of the vertebral bodies were usual, degenerative changes in the vertebral bodies were determined.

On a series of control postoperative MR tomograms, T1- and T2-weighted in two projections, a condition after removal of L5/S1 disc herniation was determined. Physiological lordosis was smoothed. The height of the intervertebral discs L4-S1 and signals from them according to T2 were reduced. Postoperative defect of the posterior structures of L5 vertebra on the right was determined.

At the level of the operated disc, fibrotic changes were determined, spreading into the intervertebral foramen, associated with which a medial protrusion of 0.3 cm was determined.

A dorsal left-sided medial-paramedial disc herniation L4/L5, 0.5 cm in size, extending into the intervertebral foramen on both sides, more on the left with significant narrowing, deforming the adjacent part of the dural sac was determined.

A dorsal diffuse protrusions of L2-L4 discs, 0.3 cm in size, extending into the intervertebral foramen on both sides was determined.

Hypertrophy of the yellow ligaments, deformity of the articular facets of the facet joints at the study levels were determined.

In the body of L4 vertebra, a section of pathological MR signal of hyperintense T2 and T1, 1.0*0.6 cm in size, of a reticular structure was determined, probably a hemangioma.

Lumen of the spinal canal was narrowed at the level of the revealed changes, signal from the spinal cord structure (T1 and T2) was not changed. Shape and size of the vertebral bodies were usual, signs of dystrophic changes in the vertebral bodies were determined.

Conclusion: MR-image of degenerative-dystrophic changes in the cervical and lumbosacral spine. Consequences of surgical treatment of herniated disc L5/C1. Hernia of L4/L5 disc is less than 6 mm. Signs of spondyloarthrosis were determined.

TABLE 5
The patient's condition before and after the treatment

| Patient of | | |
|---|---|---|
| e×ample 5 | Before the treatment | After the treatment |
| Diagnosis | sequestrated hernia of L4-L5 | hernia of L4-L5 having a |
| | having a size of up to 11 mm | size less than 6 mm |

### Example 6

### Female Patient of the Age of 29

### Before the Treatment:

A series of MRI of the lumbosacral spine were done.

Physiological lumbar lordosis was erect. The vertical axis of the spine was shifted to the right.

The size and shape of the vertebral bodies were normal.

The vertebral canal was initially narrow-up to 13 mm.

There were small areas of fatty degeneration of the bone marrow in the adjacent parts of L4-L5 vertebral bodies.

There was a decrease in the hydration and the height of intervertebral disc L4-L5.

At L4-L5 level, a median disc extrusion of 11 mm was observed, which compressed the anterior surface of the dural sac adjacent to S1 roots on both sides.

The intervertebral foramen were slightly narrowed, without compression of L4 roots.

Sagittal size of the spinal canal at this level was 5 mm, the cauda equina roots were close together.

The facet joints were irregular, the articular facets were pointed.

The Epiconus was normally located, not thickened.

Paravertebral soft tissues were not changed.

Conclusion: Degenerative-dystrophic changes in the lumbosacral spine: osteochondrosis L4-L5, extrusion of L4-L5 disc with secondary stenosis of the spinal canal, i.e. sequestrated L4-L5 hernia having a size of 11 mm.

The patient was treated with low intensity laser radiation as follows. Two 0.38×0.8 mm needles with a light guide inside (TU 9444-001-17515211-98) were introduced into the soft tissues of a patient being under local anesthesia (using 1-2 ml of SOL lidocaine 2%), one needle from the left side and the other from the right side of L4-L5. The needles were introduced at an angle of 80 degrees and a distance of about 2 cm from the spinous processes of the spine perpendicular to the midline of the spine of certain segments, to a vertical depth of about 3 cm. The light guide was connected to the laser device “Shuttle Combi IR+” having a wavelength of 0.635 microns. The exposure time was 90 minutes, with a total dose of laser radiation about 1.5 J/cm². 30 procedures were carried out within 3 months.

In addition, the patient was further treated with indirect ILBI using an aqueous carrier solution comprising NaCl, Vitamin C, Nicotinate using “Shuttle Combi IR+” with a wavelength of 0.635 μm. Indirect ILBI was carried out once in 7 days, the procedure was repeated 5 times. The exposure time was 60 minutes.

After the treatment, MRI showed the following:

Spinal MRI at Th12-S2 level did not detect bone destruction.

The spinal canal in sagittal size is from 18 mm (initially not narrowed).

The ratio of the vertebral bodies was not changed.

The lumbar lordosis was erect.

Decrease in the signal intensity in T2-WI and the height of the intervertebral discs was mainly observed at L4-L5 level.

Modic type 1 degenerative changes (mild degenerative edema) subchondral in the bodies of the adjacent vertebrae of L4-L5 segment were observed.

The facet joints were not changed.

The dorsal median extrusion of L4-L5 disc of less than 3.9 mm, without displacement, deforming the posterior longitudinal ligament was determined. Secondary spinal stenosis was determined, the sagittal size of the spinal canal was narrowed to 11 mm.

The intervertebral foramen were slightly narrowed at L4-L5 level.

The spinal cord was not changed, not compressed.

Paravertebral soft tissues were without pathology.

Conclusion: MR signs of osteochondrosis of the lumbosacral spine, complicated by herniated disc L4-L5. Discogenic relative spinal stenosis. L4-L5 disc hernia had the size of 3.9 mm, the sequester was absent. In comparison to the examination before the treatment, a positive dynamics was observed: the size of the hernia was reduced by 6 mm.

TABLE 6
The patient's condition before and after the treatment

| Patient of | | |
|---|---|---|
| e×ample 6 | Before the treatment | After the treatment |
| Diagnosis | sequestrated hernia of L4-L5 | protrusion of L4-L5 disc having a |
| | having a size of 11 mm | size of 3.9 mm, the sequester is |
| | pain in the area of innervation | absent |
| | of the left root of L5 | relief of pain syndrome |
| Symptoms | severe lower back pain; | the pain syndrome was completely |
| | lumbar ischialgia on the left | absent; |
| | (constant pain in the left leg); | the pain in the left leg disappeared; |
| | numbness in the left leg; | the sensitivity in the left leg was |
| | muscular weakness, stiffness in | restored; |
| | the lower back; | the weakness and stiffness |
| | sudden decrease in physical | disappeared; |
| | activity (“moved from chair | physical activity (long walks, active |
| | to chair only”) | rest with a child) was restored; |
| | insomnia. | healthy sleep. |

### Examples 7-18

Twelve patients (examples 7-18) of different gender, age and health conditions all having hernias were treated using the proposed method. All the patient had back pain and muscle syndrome. The duration of the treatment was 30 days.

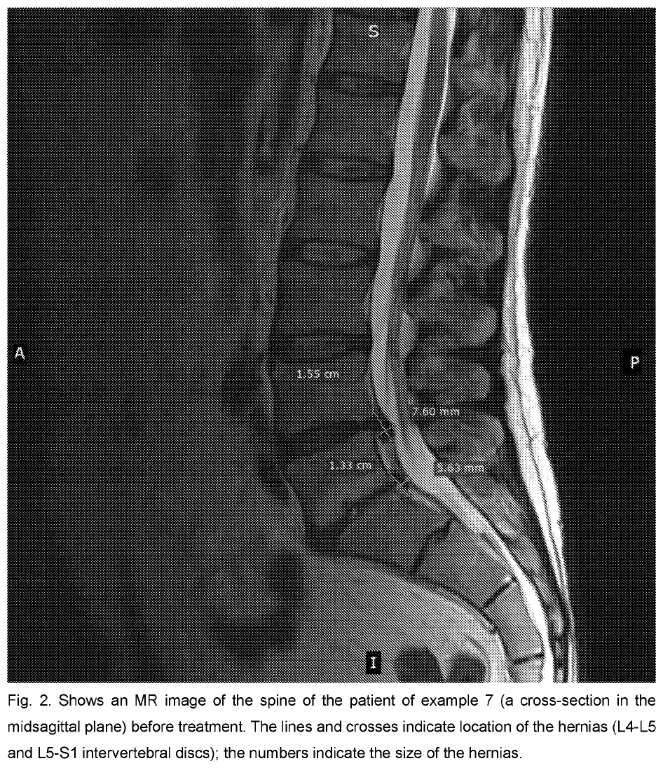

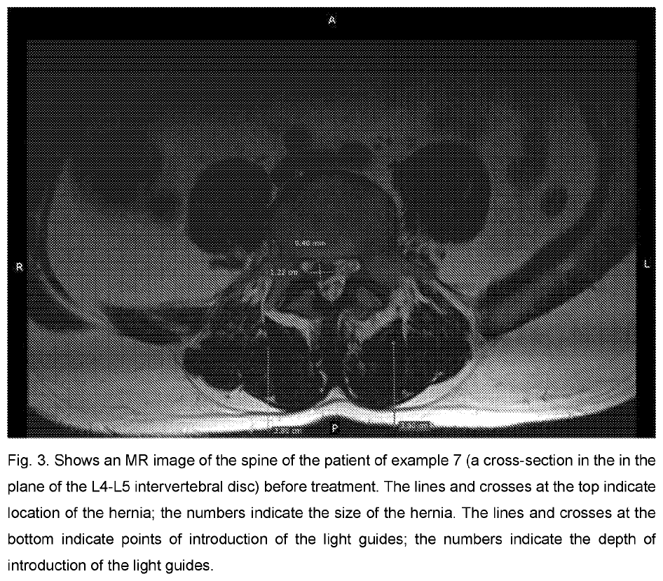

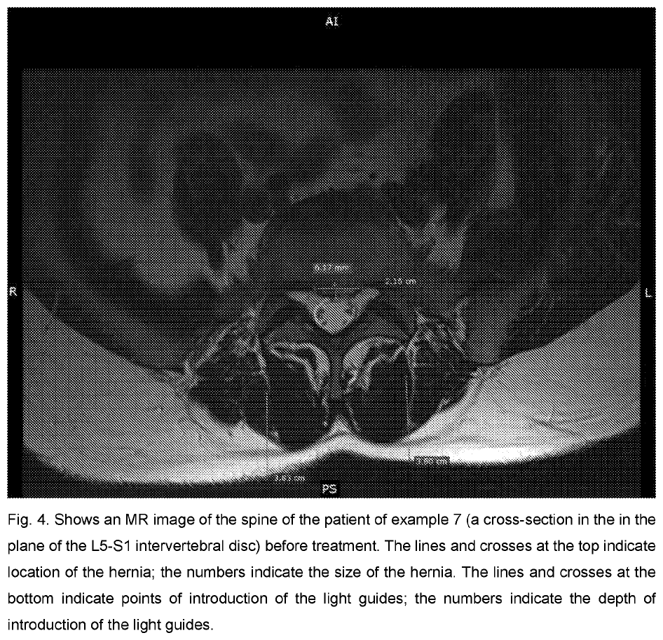

FIG. 2 shows the midsagittal plane cross-section of the spine of the patient of example 7 before the treatment, taken using MR imaging. Two hernias of the L4-L5 and L5-S1 intervertebral discs having the size of 15.5×7.6×12.2 mm 13.3×5.6×21.5 mm, respectively, are clearly seen. FIGS. 3 and 4 respectively show cross-sectional view of the L4-L5 and L5-S1 intervertebral discs of the spine of the same patient before the treatment. The two hernias are clearly seen on these figures as well. FIGS. 3 and 4 also show points of introduction of the light guides, which are located in the inner part of the dorsal paravertebral muscles.

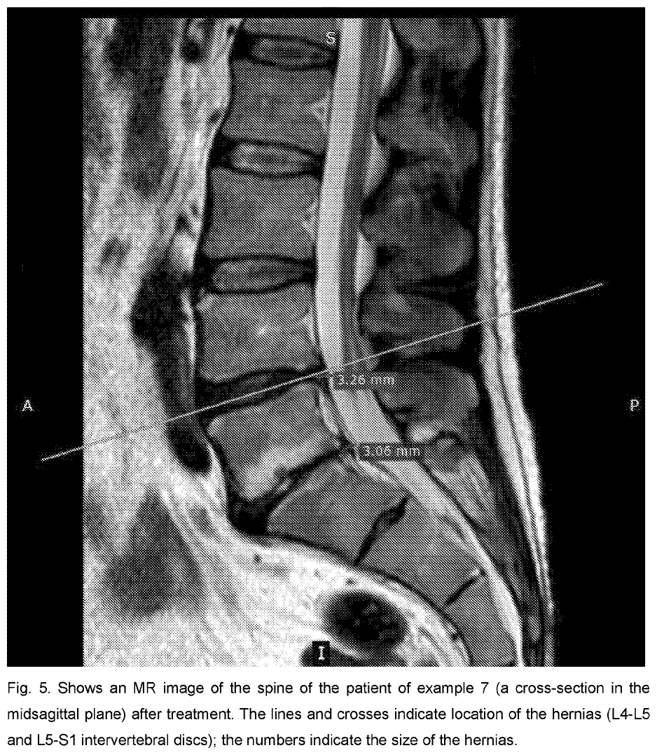

FIG. 5 shows the MR image of the spine of the patient of example 7 after 30 days of treatment. As can be seen, the hernias significantly shrank in size to 3.2 mm and 3 mm, respectively. Thus, application of the proposed method to patient of example 7 proved to be highly effective.

Similar results were obtained for the patients of examples 8-18 as can be seen from Table 7 below.

TABLE 7
Results of the treatment of the patients of e×amples 7-18

| | Location | | | Size of hernia | | | |
|---|---|---|---|---|---|---|---|
| E×ample | of Hernias | Age | Gender | Before treatment | | After treatment | |
| 7 | L4-S1 | 47 | Male | 15.5 × 7.6 × 12.2 | mm, | 3.2 mm, 3 mm | |
| | | | | 13.3 × 5.6 × 21.5 | mm | | |
| 8 | L4-S1 | 43 | Female | 6.6 mm, 12.5 mm | | 2.8 mm, 3.5 mm | |
| 9 | L4-L5 | 29 | Male | 6.7 | mm | 2.5 | mm |
| 10 | L4-S1 | 50 | Female | 14 × 6.1 × 14 | mm, | 2.3 mm, 4.3 mm | |
| | | | | 7 × 10 × 10 | mm | | |
| 11 | L3-S1 | 33 | Male | 29 × 12.9 × 17 | mm, | 25 × 6 × 13 | mm, |
| | | | | 5.4 × 15 × 10 | mm, | 4.7 × 12 × 8 | mm, |
| | | | | 6.6 × 10 × 28 | mm | 5.4 × 9 × 21 | mm |
| 12 | L4-L5 | 29 | Female | 12 × 13 × 16 | mm | 4.8 × 14 | mm |
| 13 | L4-S1 | 70 | Female | 11.3 × 10.6 × 11.5 | mm | 6.8 × 2.5 × 17.4 | mm, |
| | | | | | | 2.7 × 8.3 | mm |
| 14 | L3-S1 | 40 | Female | 13.2 × 5.1 | mm, | 10.9 × 2.9 | mm, |
| | | | | 10.9 × 6.4 | mm, | 10.2 × 3.2 | mm, |
| | | | | 10.8 × 4.8 | mm | 8.3 × 3.4 | mm |
| 15 | L4-S1 | 37 | Female | 13.7 × 4.4 × 22.3 | mm, | 3 mm, 9.9 × 5.5 mm | |
| | | | | 15.1 × 10.2 × 21 | mm | | |
| 16 | L5-S1 | 45 | Male | 13.7 × 17.5 × 22.7 | mm | 2.8 × 11.1 | mm |
| 17 | L5-S1 | 33 | Male | 10.6 × 12.7 × 19 | mm | 11 × 4.9 × 8.3 | mm |
| 18 | L5-S1 | 58 | Male | 27 × 10 × 16 | mm | 10 × 3 × 14 | mm |

The above examples demonstrate that application of the proposed method provides significant improvement to the health condition of the patient having hernia of intervertebral disc.

1. A method of treating a hernia of an intervertebral disc in a patient, the method comprising introducing at least one light guide into the patient's soft tissue, surrounding the hernia, and subjecting the soft tissue to a laser radiation using said light guide, the laser radiation having a wavelength in the range of from 625 to 740 nm and a power in the range of from 1 and 100 mW.

2. The method according to claim 1, wherein the wavelength of the laser radiation is from 630 to 700 nm, preferably from 630 to 680 nm, more preferably from 630 to 650 nm.

3. The method according to claim 1 or 2, wherein the power of the laser radiation is from 1 to 50 mW, preferably from 3 to 20 mW, more preferably from 6 to 15 mW.

4. The method according to any one of claims 1 to 3, wherein the time of subjecting the soft tissue to the laser radiation is from 1 to 500 min, preferably from 30 to 100 min, more preferably from 30 to 90 min.

5. The method according to any one of claims 1 to 4, wherein the light guide is introduced into the soft tissue in the plane of the intervertebral disc being treated±0.5 cm, in the anterior direction at a distance from 0.5 to 1.5 cm away from the intervertebral joints.

6. The method according to any one of claims 1 to 5, wherein the light guide is introduced into the soft tissue at a distance from 1.5 to 2.5 cm away from the midsagittal plane passing through the spinous processes.

7. The method according to any one of claims 1 to 6, wherein the soft tissue is the muscle tissue.

8. The method according to claim 7, wherein the muscle tissue is the tissue of the dorsal paravertebral muscles.

9. The method according to claim 8, wherein the light guide is introduced into the tissue of the dorsal paravertebral muscles at a distance at least 1.0-1.5 cm away from the border of the dorsal paravertebral muscles with the thoracolumbar fascia.

10. The method according to any one of claims 1 to 9, wherein more than one hernias are subjected to laser radiation simultaneously, in particular from 2 to 4 hernias are subjected to laser radiation simultaneously.

11. The method according to any one of claims 1 to 10, wherein the at least one light guide is introduced at both sides of the midsagittal plane.

12. The method according to any one of claims 1 to 11, wherein a single hernia is subjected to the laser radiation using more than one light guide, in particular, from 2 to 8 light guides.

13. The method according to any one of claims 1 to 12, the steps of which are repeated several times, in particular, from 10 to 40 times, 3-7 times a week.

14. The method according to any one of claims 1 to 13, further comprising a step of indirect intravascular laser blood irradiation.

15. The method according to claim 14, wherein the step of indirect intravascular laser blood irradiation is carried out by subjecting a carrier solution to a laser radiation and simultaneously introducing the carrier solution being irradiated into the patient's vascular system.

16. The method according to any one of claims 14 to 15, wherein the laser radiation at the step of indirect intravascular laser blood irradiation has a wavelength in the range of from 625 to 740 nm and a power in the range of from 1 to 15 mW and is applied during a time period of from 5 to 120 min.

17. The method according to any one of claims 15 to 16, wherein the carrier solution comprises NaCl, nicotinate, vitamin C, and water.

18. The method according to claims 14 to 17, wherein the step of indirect intravascular laser blood irradiation is repeated several times, in particular, from 2 to 7 times, once in 5-10 days.

19. The method according to any one of claims 1 to 18, further comprising a step of direct intravascular laser blood irradiation.

20. The method according to claim 19, wherein the step of indirect intravascular laser blood irradiation is carried out by directly subjecting the patient's blood to a laser radiation.

21. The method according to any one of claims 19 to 20, wherein the laser radiation at the step of direct intravascular laser blood irradiation has a wavelength in the range of from 625 to 740 nm and a power in the range of from 1 to 15 mW, more preferably in the range of from 1 to 7 mW, even more preferably in the range of from 1.5 to 3 mW, and is applied during a time period of from 5 to 120 min.

22. The method according to claims 19-21, wherein the step of direct intravascular laser blood irradiation is repeated several times, in particular, from 2 to 7 times, once in 5-10 days.

23. The method according to any one of claims 1 to 22, wherein the hernia has a size in the range of from 1 to 40 mm.

24. The method according to any one of claims 1 to 23, wherein the hernia of intervertebral disc is a sequestered hernia of the intervertebral disc.

25. The method according to any one of claims 1 to 24, wherein the light guide is introduced inside a needle.

26. A radiating system for treating a hernia of an intervertebral disc in a patient, the system comprising:
a radiating source operable to generate a laser radiation having a wavelength in the range of from 625 to 740 nm; wherein
the radiating source is configured to be connected with at least one light guide for delivering the laser radiation into the patient's soft tissue, surrounding the hernia, with a power in the range of from 1 and 100 mW.

27. The radiating system according to claim 26, wherein the power of the laser radiation delivered into the patient's soft tissue is from 1 to 50 mW, preferably from 3 to 20 mW, more preferably from 6 to 15 mW.

28. A radiating system for treating a hernia of an intervertebral disc in a patient, the system comprising:
a radiating source operable to generate a laser radiation having a wavelength in the range of from 625 to 740 nm and a power in the range of from 1.5 and 150 mW; wherein
the radiating source is configured to be connected with at least one light guide for delivering the laser radiation into the patient's soft tissue, surrounding the hernia.

29. The radiating system according to claim 28, wherein the power of the laser radiation generated by the radiation source is from 1.1 to 75 mW, preferably from 4 to 30 mW, more preferably from 7 to 25 mW.

30. The radiating system according to any one of claims 26 to 29, wherein the wavelength of the laser radiation is from 630 to 700 nm, preferably from 630 to 680 nm, more preferably from 630 to 650 nm.

31. The radiating system according to any one of claims 26 to 30, capable of generating a laser radiation for a time from 1 to 500 min, preferably from 30 to 100 min, more preferably from 30 to 90 min.

32. The radiating system according to any one of claims 26 to 31, wherein the light guide is for delivering the laser radiation into the soft tissue in the plane of the intervertebral disc being treated±0.5 cm, in the anterior direction at a distance from 0.5 to 1.5 cm away from the intervertebral joints.

33. The radiating system according to any one of claims 26 to 32, wherein the light guide is for delivering the laser radiation into the soft tissue at a distance from 1.5 to 2.5 cm away from the midsagittal plane passing through the spinous processes.

34. The radiating system according to any one of claims 26 to 33, wherein the soft tissue is the muscle tissue.

35. The radiating system according to claim 34, wherein the muscle tissue is the tissue of the dorsal paravertebral muscles.

36. The radiating system according to claim 35, wherein the light guide is for delivering the laser radiation into the tissue of the dorsal paravertebral muscles at a distance at least 1.0-1.5 cm away from the border of the dorsal paravertebral muscles with the thoracolumbar fascia.

37. The radiating system according to any one of claims 26 to 36, wherein the radiation source is configured to be connected with more than one light guide, in particular, with from 2 to 8 light guides.

38. The radiating system according to any one of claims 26 to 37, configured for use for indirect intravascular laser blood irradiation.

39. The radiating system according to claim 38, wherein the use for indirect intravascular laser blood irradiation comprises subjecting a carrier solution to a laser radiation and simultaneously introducing the carrier solution being irradiated into the patient's vascular system.

40. The radiating system according to claim 39, wherein the use for indirect intravascular laser blood irradiation comprises subjecting a carrier solution to a laser radiation having a wavelength in the range of from 625 to 740 nm and a power in the range of from 1 to 15 mW and for a time period of from 5 to 120 min.

41. The radiating system according to any one of claims 39 to 40, wherein the carrier solution comprises NaCl, nicotinate, vitamin C, and water.

42. The radiating system according to any one of claims 26 to 41, configured for use for direct intravascular laser blood irradiation.

43. The radiating system according to claim 42, wherein the use for direct intravascular laser blood irradiation comprises directly subjecting the patient's blood to a laser radiation having a wavelength in the range of from 625 to 740 nm and a power in the range of from 1 to 15 mW, more preferably in the range of from 1 to 7 mW, even more preferably in the range of from 1.5 to 3 mW, for a time period of from 5 to 120 min.

44. The radiating system according to any one of claims 26 to 43, wherein the hernia has a size in the range of from 1 to 40 mm.

45. The radiating system according to any one of claims 26 to 44, wherein the hernia of the intervertebral disc is a sequestered hernia of the intervertebral disc.

46. The radiating system according to any one of claims 26 to 45, wherein the light guide is disposed inside a needle.

47. Use of the radiating system according to any one of claims 26 to 46 for treating a hernia of an intervertebral disc in a patient.
